(19) United States
(12) Patent Application Publication
(10) Pub. No.: US 2008/0234578 A1
(43) Pub. Date: Sep. 25, 2008

(54) Multi-modality mammography reconstruction method and system

(75) Inventor: Bernhard Erich Hermann Claus, Niskayuna, NY (US)

Correspondence Address: GENERAL ELECTRIC COMPANY (PCPI);C/O FLETCHER YODER, P. O. BOX 692289, HOUSTON, TX, 77269-2289, US

(73) Assignee: General Electric Company

(21) Appl. No.: 11/725,386

(22) Filed: Mar. 19, 2007

(51) Int. Cl. A61B 8/00

(52) U.S. Cl. 600/437

(57) ABSTRACT

A method, system, and software are provided for joint reconstruction of three-dimensional images using multiple imaging modalities. In an exemplary embodiment, the present approach includes providing a first dataset acquired via a first imaging technique or a first image generated from the first dataset, providing a second dataset acquired via a second imaging technique or a second image generated from the second dataset, and generating a volumetric dataset by extracting information from the first and second datasets or images. The first imaging technique may have better resolution than the second imaging technique in a first direction, and the second imaging technique may have better resolution than the first imaging technique in a second direction. There is provided a system and one or more tangible, machine readable media for performing the act of generating the volumetric dataset by extracting information from the first and second datasets or images.

### BACKGROUND

The present approach relates generally to the field of medical imaging, and more specifically to the fields of tomosynthesis and ultrasound imaging. In particular, the present approach relates to the combination of data acquired during tomosynthesis and ultrasound.

In modern healthcare facilities, medical diagnostic and imaging systems are used for identifying, diagnosing, and treating diseases. Diagnostic imaging refers to any visual display of structural or functional patterns of organs or tissues for a diagnostic evaluation. Currently, a number of modalities exist for medical diagnostic and imaging systems. These include, for example, ultrasound systems, X-ray imaging systems (including tomosynthesis systems), molecular imaging systems, computed tomography (CT) systems, positron emission tomography (PET) systems and magnetic resonance imaging (MRI) systems.

One such imaging technique is tomosynthesis, in which X-ray attenuation data is obtained for a region of interest over a limited angular range and used to construct volumetric or generally three-dimensional images. For example, tomosynthesis may be employed to acquire mammography information whereby a breast of a patient may be non-invasively examined or screened to visualize and detect abnormalities, such as lumps, fibroids, lesions, calcifications, and so forth. Such X-ray imaging and tomosynthesis systems are generally effective for detailed characterization of benign and cancerous structures such as calcifications and masses embedded in the breast tissue.

Another known imaging technique is ultrasound. An ultrasound imaging system uses an ultrasound probe for transmitting ultrasound signals into an object, such as the breast of the patient being imaged, and for receiving reflected ultrasound signals there from. The reflected ultrasound signals received by the ultrasound probe are processed to reconstruct an image of the object. Ultrasound imaging is useful as an alternate tool for diagnosis, such as for differentiating benign cysts and masses.

Generally, when such tomosynthesis and ultrasound data are collected for a given volume, the resulting images are collected and analyzed independently. At best, the images are compared side-by-side to determine if any abnormalities seen in images produced using one modality are also present in images produced using the other modality. However, there is complementary information in the tomosynthesis and ultrasound datasets, not only concerning different tissue characteristics that are made visible though the use of these different modalities, but also in terms of the inherent resolution exhibited by these imaging systems. In particular, tomosynthesis imaging exhibits a poor depth resolution in combination with a very good in-plane resolution, while ultrasound imaging exhibits a good depth-resolution combined with a somewhat reduced in-plane resolution.

### BRIEF DESCRIPTION

There is provided a method for generating an imaging dataset including providing a first dataset acquired via a first imaging technique or a first image generated from the first dataset, providing a second dataset acquired via a second imaging technique or a second image generated from the second dataset, and generating a volumetric dataset by extracting information from the first and second datasets or images. The first imaging technique may have better resolution than the second imaging technique in a first direction and the second imaging technique may have better resolution than the first imaging technique in a second direction.

There is further provided tangible, machine readable media, with code executable to perform the act of generating a volumetric dataset by extracting information from a first dataset acquired using a first imaging technique or a first image generated from the first dataset and a second dataset acquired using a second imaging technique or a second image generated from the second dataset. The first imaging technique may have better resolution than the second imaging technique in a first direction and the second imaging technique may have better resolution than the first imaging technique in a second direction.

In addition, there is provided a system including a computer configured to generate a volumetric dataset by extracting information from a first dataset acquired using a first imaging technique or a first image generated from the first dataset and a second dataset acquired using a second imaging technique or a second image generated from the second dataset. The first imaging technique may have better resolution than the second imaging technique in a first direction and the second imaging technique may have better resolution than the first imaging technique in a second direction.

### DRAWINGS

These and other features, aspects, and advantages of the present approach will become better understood when the following detailed description is read with reference to the accompanying drawings in which like characters represent like parts throughout the drawings, wherein:

FIG. 1 is a diagrammatic representation of one embodiment of a mammography imaging system in accordance with aspects of the present approach;

FIG. 2 is a diagrammatic representation of one embodiment of an ultrasound imaging system in accordance with aspects of the present approach; and

FIGS. 3-7 are flow charts illustrating exemplary embodiments or aspects of the present approach.

### DETAILED DESCRIPTION

The present approach is directed towards joint reconstruction of images with better resolutions in different directions. For example, tomosynthesis and ultrasound images may advantageously be combined in a joint reconstruction to leverage the better in-plane resolution in tomosynthesis and the better resolution in the direction of wave propagation in ultrasound. In the simplest embodiment, images acquired with different techniques or modalities may have different resolution characteristics in different orthogonal directions, such as the X, Y, and Z planes, however it should be understood that the present approach is not limited to these cases. In other examples, a cranio-caudal (CC) tomosynthesis image may be combined with a medio-lateral oblique (MLO) tomosynthesis image in an improved joint reconstruction according to the present approach. Likewise, one or more conventional mammography images or single X-ray projection images may be used as one of the modalities according to the present approach. In addition, the present approach need not be limited to joint reconstruction of images acquired using two techniques but may be applied to images acquired using more than two techniques. For example, a MLO tomosynthesis image, a CC tomosynthesis image, and an ultrasound image may be combined in a three-way joint reconstruction. This approach may be applied to the field of mammography, where improved imaging is needed to provide improved sensitivity and specificity through early detection of malignant growths and to improve the correct classification of imaged structures by reducing the rate of incorrect classifications of benign cysts and masses. However, as will be appreciated by those of ordinary skill in the art, the present approach may also be applied in other medical and non-medical contexts.

The present specification describes the use of tomosynthesis and ultrasound as exemplary imaging modalities. However, it should be appreciated that the present approach may employ other imaging modalities or the same type of imaging modality operated using different scan parameters, protocols, trajectories, or orientations which result in the acquisition of image data that has different resolution characteristics in different directions. For convenience, the term imaging technique will be used herein to describe the acquisition of images using a given modality and/or a given configuration, such as a given orientation, that results in image data being acquired with resolution characteristics that are better in one direction relative to another direction. For example, acquisition of breast images using a tomosynthesis system and an ultrasound system from the same orientation constitute two distinct imaging techniques due to the distinctly separate imaging modalities and due to the different resolution characteristics of these modalities. For instance, image data acquired at a given orientation by an ultrasound system may have superior resolution in the wave-propagation direction relative to images acquired by a tomosynthesis system with the breast at the same orientation. Conversely, images acquired by the tomosynthesis system may have superior in-plane resolution (i.e., parallel to a detector) than images acquired by the ultrasound system with the breast at the same orientation. Further, a single imaging modality employed at different orientations or using different scan parameters or configurations may be considered as constituting two distinct imaging techniques, as used herein. For example, using a tomosynthesis system to acquire breast images in a CC orientation and in a MLO orientation constitute separate imaging techniques due to the different resolution characteristics in the acquired image data, i.e., the “in-plane” image data for each of these techniques is essentially orthogonal. With this clarification that an imaging technique, as used herein, encompasses both images acquired using different modalities (at the same or different orientations) or the same modality but at different orientations or using different scan parameters or configurations, the following discussion is provided.

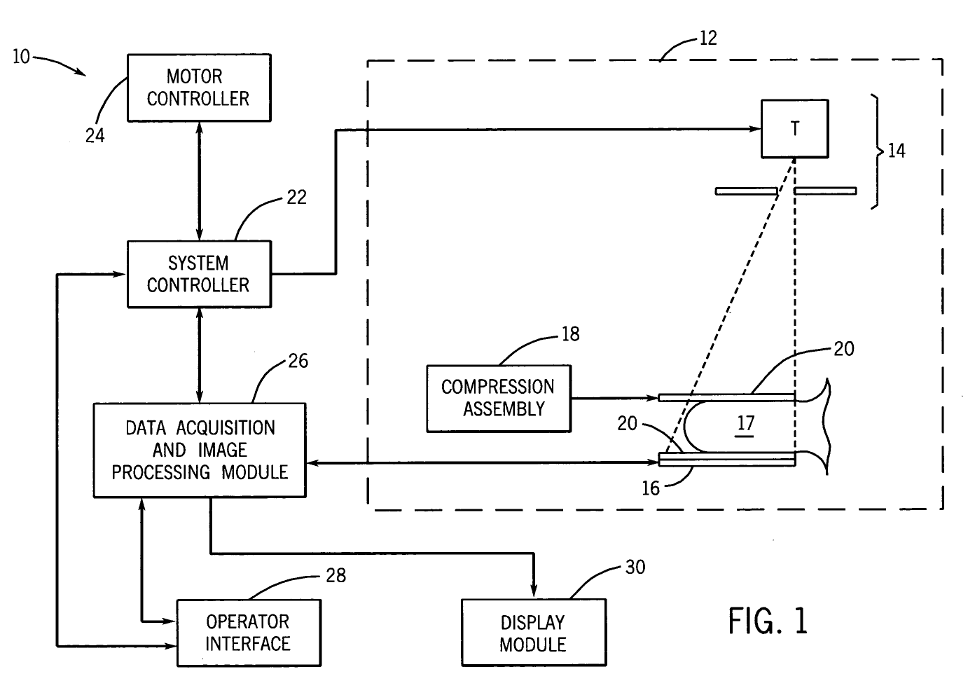

Turning now to the drawings, and referring first to FIG. 1, an exemplary tomosynthesis imaging system 10 for use in accordance with the present approach is illustrated diagrammatically. As depicted, the tomosynthesis imaging system 10 includes an image data acquisition system 12. The image data acquisition system 12 includes an X-ray source 14, an X-ray detector 16 and a compression assembly 18. The tomosynthesis imaging system 10 further includes a system controller 22, a motor controller 24, data acquisition and image-processing module 26, an operator interface 28 and a display module 30.

The X-ray source 14 further includes an X-ray tube and a collimator configured to generate a beam of X-rays when activated. The X-ray tube is one example of the X-ray source 14. Other types of the X-ray sources 14 may include solid state X-ray sources having one or more emitters. The X-ray source 14 may be movable in one, two or three dimensions, either by manual or by automated means. The image data acquisition system 12 may move the X-ray source 14 via tracks, ball-screws, gears, belts, and so forth. For example, the X-ray source 14 may be located at an end of a mechanical support, such as a rotating arm or otherwise adjustable support, which may be moved by the image data acquisition system 12 or by an operator. Instead of, or in combination with, a mechanical displacement of the X-ray source 14, different view angles may be achieved through individually addressable source points.

The X-ray detector 16 may be stationary, or may be configured to move either independently or in synchrony with the X-ray source 14. In a present embodiment, the X-ray detector 16 is a digital flat panel detector. The image data acquisition system 12 may move the X-ray detector 16, if mobile, via tracks, ball-screws, gears, belts, and so forth. In one embodiment, the X-ray detector 16 also provides support for an object, such as a breast 17 of a patient to be imaged, thereby forming one part of the compression assembly 18. In other embodiments, the X-ray detector may be disposed immediately or proximately beneath a bottom plate of compression assembly 18, i.e., in such an embodiment, the breast 17 does not rest directly on the detector 16 but on a plate or other compressing support above the detector 16.

The compression assembly 18, whether including two compression plates or a compression plate and the detector 16, is configured to compress the patient breast 17 for performing tomosynthesis imaging and to stabilize the breast 17 during the imaging process to minimize patient motion while data is acquired. In one embodiment, the breast is compressed to near uniform thickness. In the depicted embodiment, the compression assembly 18 includes at least one mammography compression plate 20, which may be a flat, inflexible plate, deformable sheet, or alternative compression device. In one embodiment, the mammography compression plate 20 is configured to be radiolucent to transmit X-rays and is further configured to be sonolucent to transmit ultrasound signals. The compression assembly 18 may be used to stabilize the imaged breast 17 during acquisition of both the tomosynthesis and the ultrasound datasets, thereby enabling the acquisition of co-registered tomosynthesis X-ray images, ultrasound images, and Doppler images.

The system controller 22 controls operation of the image data acquisition system 12 and provides for any physical motion of the X-ray source 14 and/or the X-ray detector 16. In the depicted embodiment, movement is, in turn, controlled through the motor controller 24 in accordance with an imaging trajectory for use in tomosynthesis. Therefore, by means of the image data acquisition system 12, the system controller 22 may facilitate acquisition of radiographic projections at various angles relative to a patient. The system controller 22 further controls an activation and operation of other components of the system, including collimation of the X-ray source 14. Moreover, the system controller 22 may be configured to provide power and timing signals to the X-ray source 14. The system controller 22 may also execute various signal processing and filtration functions. In general, the system controller 22 commands operation of the tomosynthesis imaging system 10 to execute examination protocols and to acquire resulting data.

For example, in the depicted embodiment, the system controller 22 controls a tomosynthesis data acquisition and image-processing module 26. The tomosynthesis data acquisition and image-processing module 26 communicates with the X-ray detector 16 and typically receives data from the X-ray detector 16, such as a plurality of sampled analog signals or digitized signals resulting from exposure of the X-ray detector to X-rays. The tomosynthesis data acquisition and image-processing module 26 may convert the data to digital signals suitable for processing and/or may process sampled digital and/or analog signals to generate volumetric images of the breast 17.

The operator interface 28 may include a keyboard, a mouse, and other user interaction devices. The operator interface 28 can be used to customize settings for the tomosynthesis imaging and for effecting system level configuration changes as well as for allowing operator activation and operation of the tomosynthesis imaging system 10. In the depicted embodiment, the operator interface 28 is connected to the tomosynthesis data acquisition and image-processing module 26, the system controller 22 and the display module 30. The display module 30 presents a reconstructed image of an object, or of a region of interest within the object, based on data from the data acquisition and image-processing module 26. As will be appreciated by those skilled in the art, digitized data representative of individual picture elements or pixels is processed by the tomosynthesis data acquisition and image-processing module 26 to reconstruct the desired image. The image data, in either raw or processed forms, may be stored in the system or remotely for later reference and image reconstruction.

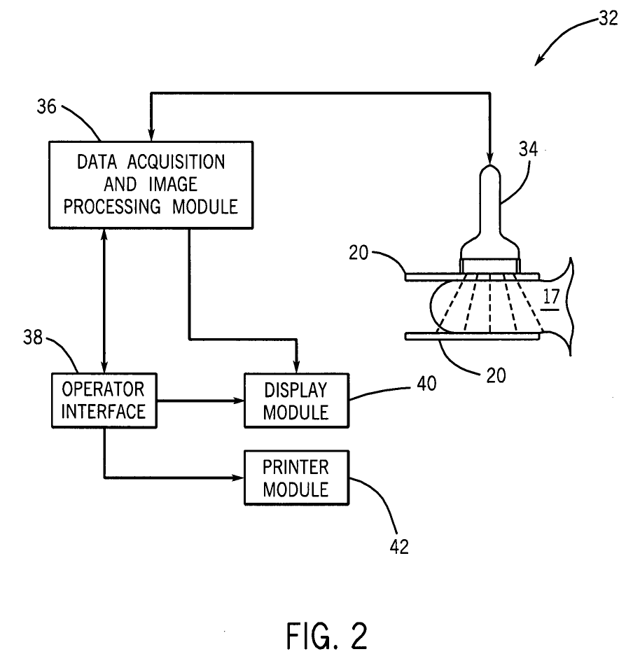

FIG. 2 illustrates an exemplary ultrasound imaging system 32 for use in conjunction with the present approach. As depicted, the ultrasound imaging system 32 includes an ultrasound probe 34, an ultrasound data acquisition and image-processing module 36, which includes beam-formers and image reconstruction and processing circuitry, an operator interface 38, a display module 40 and a printer module 42. In a hybrid imaging system based upon both X-ray and ultrasound techniques, certain of these components or modules may be partially or fully integrated to perform image acquisition and processing for both systems.

The ultrasound imaging system 32 uses the ultrasound probe 34 for transmitting a plurality of ultrasound signals into an object, such as the breast 17 of a patient being imaged, and for receiving a plurality of reflected ultrasound signals therefrom. The ultrasound probe 34, according to aspects of the present approach, includes at least one transducer for generating ultrasound waves or energy from mechanical or electromechanical impulses and vice versa. As will be appreciated by those of ordinary skill in the art, the plurality of reflected ultrasound signals from the object carry information about thickness, size, and location of various tissues, organs, tumors, and anatomical structures in relation to transmitted ultrasound signals. The plurality of reflected ultrasound signals received by the ultrasound probe 34 are processed for constructing an image of the object. In certain embodiments, the ultrasound probe 34 can be hand-held or mechanically positioned using a robotic assembly. The ultrasound imaging system 32 may also incorporate beam steering technology to reach all areas of the imaged breast. In addition, according to an embodiment of the present approach, the ultrasound imaging system 32 may use compounding, that is, a suitable combination of signals from the same area of the breast 17 that leads to improved ultrasound image quality.

The ultrasound data acquisition and image-processing module 36 sends signals to and receives information from the ultrasound probe 34. Thus, the ultrasound data acquisition and image-processing module 36 controls strength, beam focus or forming, duration, phase, and frequency of the plurality of ultrasound signals transmitted by the ultrasound probe 34, and decodes the information contained in the plurality of reflected ultrasound signals from the object to a plurality of discernable electrical and electronic signals. Once the information is obtained, an ultrasound image of the object located within a region of interest is reconstructed in accordance with generally known reconstruction techniques.

The operator interface 38 may include a keyboard, a mouse, and other user interaction devices. The operator interface 38 can be used to customize a plurality of settings for an ultrasound examination, to effect system level configuration changes, and to allow operator activation and operation of the ultrasound imaging system 32. The operator interface 38 is connected to the ultrasound data acquisition and image-processing module 36, the display module 40 and to the printer module 42. The display module 40 receives image information from the ultrasound data acquisition and image-processing module 36 and presents the image of the object within the region of interest of the ultrasound probe 34. The printer module 42 is used to produce a hard copy of the ultrasound image in either gray-scale or color. As noted above, some or all of these system components may be integrated with those of the tomosynthesis X-ray system described above.

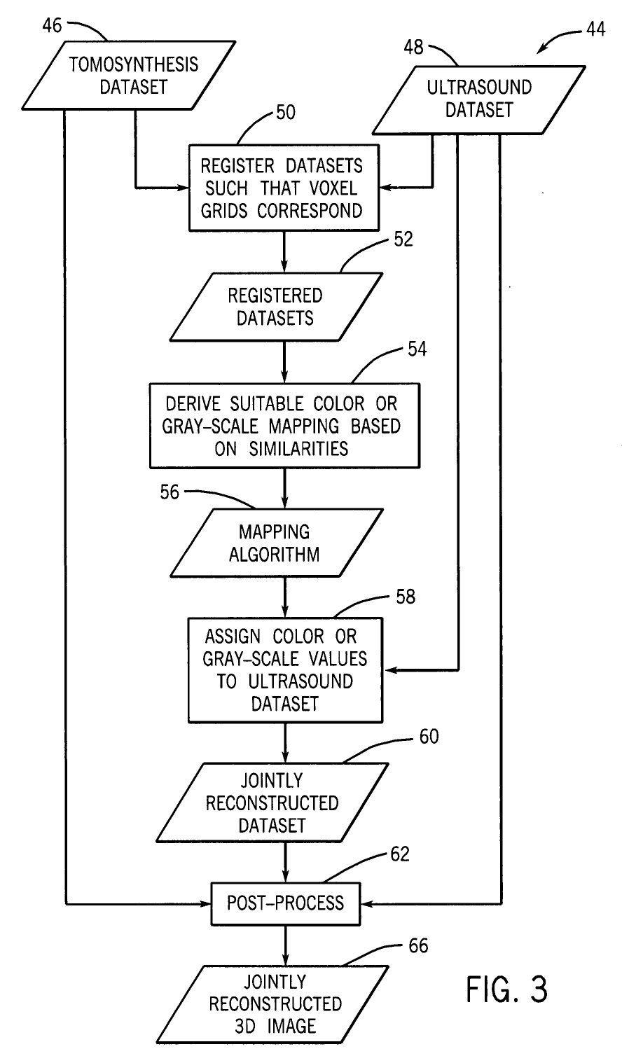

Turning now to FIG. 3, an exemplary embodiment of the present approach is illustrated in a flow chart. At least one tomosynthesis dataset 46 may be acquired via the system described in reference to FIG. 1 or via an alternate tomosynthesis imaging system. Likewise, at least one ultrasound dataset 48 may be acquired via the system described in reference to FIG. 2 or via an alternate ultrasound imaging system. Alternatively, the present approach may be applied to previously-acquired tomosynthesis and/or ultrasound data. Raw data from the tomosynthesis and ultrasound imaging systems may have been processed to produce volumetric datasets 46 and 48. For example, the tomosynthesis dataset 46 may have been suitably reconstructed from a set of individual projection images that were gain-corrected, log-corrected, or corrected for some geometrical effects, such as path length between source and each pixel, effective pixel area, or path length through tissue. In addition, the tomosynthesis projection data may have been scatter corrected or may be virtually scatter-free, such as in slot-scanning systems.

In an exemplary process 44, at least one tomosynthesis dataset 46 and at least one ultrasound dataset 48 may be registered in a step 50. In this step 50, the datasets 46 and 48 may be aligned such that their respective coordinate systems correspond. The registration may be rigid or non-rigid, with varying degrees of flexibility. Depending on the resolution of the datasets 46 and 48, the registration may also include an interpolation step, such as, for example, tri-linear interpolation or nearest neighbor interpolation, to map both datasets to the same voxel grid. In cases where the datasets are acquired together they may be intrinsically registered to one another, in which case the registration step 50 may be omitted or only an interpolation may be performed. In illustrations of further embodiments of the present approach this registration step is omitted, however it should be understood that registration may be required if the tomosynthesis and ultrasound datasets are not intrinsically registered. This may be especially important in situations where the imaged breast is not in the same position while the two datasets are acquired.

Registered datasets 52 may be compared to one another to derive a suitable color or gray-scale mapping in a step 54. This derivation may employ a method, such as mutual information, wherein some similarity criterion between the datasets is minimized. The mapping function may be one-to-one, where any gray value in the ultrasound dataset corresponds to a single associated attenuation value in the tomosynthesis dataset and vice versa, many-to-one, where more than one gray value in one dataset may be assigned to a single gray value in the other dataset, one-to-many, or many-to-many. A mapping algorithm 56 may be derived such that each color or gray-scale value represented in the ultrasound dataset can be assigned a corresponding X-ray attenuation value, where the assigned attenuation value is derived from the mapping between the tomosynthesis and the ultrasound dataset. Once the mapping algorithm 56 is derived, it may be applied to the ultrasound dataset 48 in a step 58.

The resulting jointly reconstructed dataset 60 may go through a post-processing step 62. This step 62 may include, for example, coloring (i.e., assigning gray-scale or color values to voxels) the jointly reconstructed dataset 60 such that both ultrasound and X-ray characteristics of the imaged anatomy are properly represented. For example, if two regions “look” different in the ultrasound dataset 48 but are mapped to the same X-ray attenuation value, such as when the ultrasound to X-ray gray-scale mapping is many-to-one, then the regions may be represented by different colors in post-processing step 62. In one embodiment of the present approach, the jointly reconstructed dataset 60 may be represented in gray-scale values while complementary information from the ultrasound dataset may be overlaid in colors. In another embodiment of the present approach, post-processing step 62 may include reconstructing fine X-ray detail by using, for example, sparseness of data and non-linear techniques such as order statistics-based reconstruction (OSBR). In OSBR, the image data from the projection images is backprojected, then combined. Unlike in simple backprojection, where the backprojected values at each voxel are combined using an averaging operator, the backprojected values in OSBR are combined, for example, by using a voting scheme. That is, if more than half of the backprojected values indicate that the gray level in a position should be higher, then it is increased correspondingly. In another example, the reconstructed voxel value is generated as the average of all backprojected values with the exception of some of the largest and smallest values. Other order-statistics based operators may be used as well, such as median and mode. Other suitable techniques to combine the backprojected data may also be used. The sparseness of the residual projection data after re-coloring the ultrasound dataset can then be used to effectively “place” voxels of certain types of tissue at the correct locations in the dataset 60, thereby improving the resolution within the reconstructed dataset 60. In addition, the post-processing step 62 may include preparing the jointly reconstructed dataset 60 for display and displaying the jointly reconstructed three-dimensional image 66.

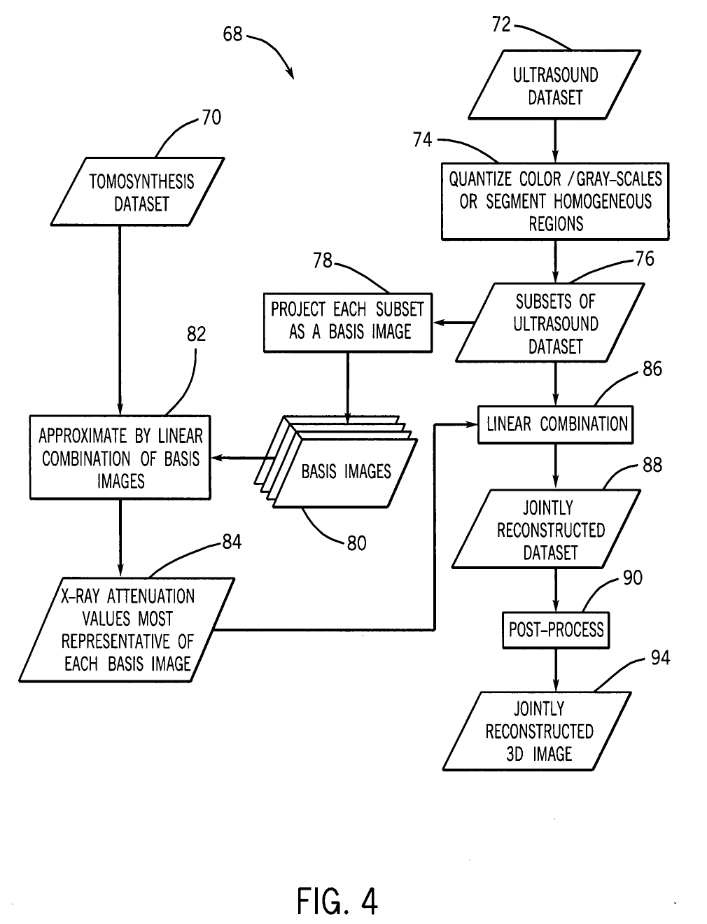

In another exemplary embodiment of the present approach, illustrated in FIG. 4, at least one tomosynthesis projection dataset 70 and at least one ultrasound dataset 72 are used as input in a process 68. Ultrasound dataset 72 may be processed in a step 74 such that subsets 76 of the ultrasound dataset are specified. This processing may be a quantization, in which the registered ultrasound dataset 72 is divided into discrete ranges of color or gray-scale values. For example, one range may include gray-scale values from 0.5 to 0.6. In this example, all voxels of the registered ultrasound dataset 72 which have a gray-scale value from 0.5 to 0.6 would be grouped into a single subset 76. This quantization may cover the entire range of gray-scale values present in the registered ultrasound dataset 72 such that every voxel is placed into a subset 76, or the quantization may apply only to gray-scale values which are present in medically relevant sections of the registered ultrasound dataset 72. The gray-scale levels that separate the different ranges of values, as well as the number of different ranges of values, may be adaptively chosen (e.g., by using suitable clustering techniques). They may also be chosen based on prior knowledge of the imaging physics. They may also be chosen manually, or in a semi-automatic fashion. The same technique may be applied to colored ultrasound data. Alternatively, the processing step 74 may include segmenting the registered ultrasound dataset 72 into homogeneous regions based on texture or visible edges according to techniques known in the art and assigning a different label to each segment. The term “homogeneous” may refer to image gray-scale or color values, as well as tissue-type characteristics (which may be reflected, e.g., in homogeneous properties of the image texture). Each homogeneous region may then be a subset 76 of the ultrasound dataset. In one embodiment of the present approach, processing step 74 may include over-segmentation such that there is a high confidence that data within each region is homogenous.

In a step 78, all locations or voxels within a subset 76 of the ultrasound dataset are assigned a value of one while locations or voxels within all other subsets 76 are assigned a value of zero, and the corresponding volume is then projected according to the tomosynthesis acquisition geometry in order to form a basis image 80. Each basis image 80 may be a family of images, including one image for each projection angle in the tomosynthesis mode. Alternatively, the basis image 80 may be a subset of all of the images or a single image. The registered tomosynthesis projection dataset 70 is then approximated by linear combination or weighted sum of the basis images 80 in a step 82. That is, each basis image 80 is assigned a weight such that the weighted sum of all the basis images 80 is approximately equal to the tomosynthesis projection dataset 70. These weights may then represent the X-ray attenuation values 84 most representative of each basis image 80. The derived X-ray attenuation values 84 may then be applied to the ultrasound subsets 76 in order to form a linear combination of the subsets 76 in a step 86. The dataset created in this linear combination step 86 represents a jointly reconstructed dataset 88 in which each quantized or segmented subset 76 of the ultrasound dataset has been assigned an X-ray attenuation value corresponding to the registered tomosynthesis dataset 70. The resulting jointly reconstructed dataset 88 may be post-processed in a step 90 using techniques similar to that of post-processing step 62. Finally, a jointly reconstructed three-dimensional image 94 may be generated or displayed.

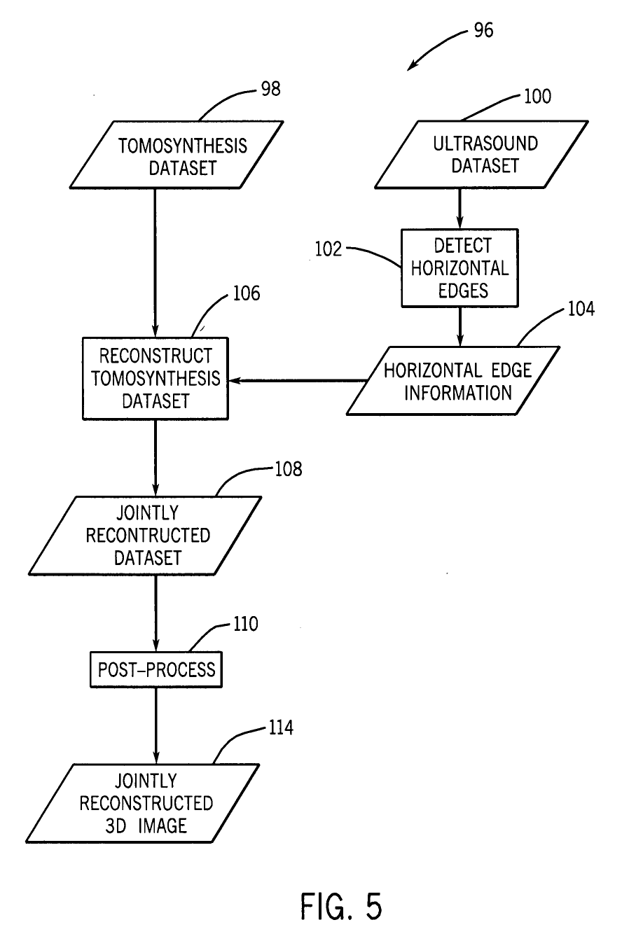

Turning now to FIG. 5, an exemplary embodiment of the present approach designated as process 96 is illustrated in a flow chart. At least one ultrasound dataset 100 may be analyzed to detect horizontal edges in a step 102. In this technique, “horizontal” means in a direction generally orthogonal to the direction of wave propagation as described above in reference to FIG. 2. The horizontal edges correspond to discontinuities in depth relative to the ultrasound probe. The orientation of the horizontal edges does not need to be strictly horizontal, but could include any orientation that may be roughly aligned with this orientation. In its most general embodiment, any edge orientation may be used. In practice, the “horizontal” plane will often be parallel to the compression plates 20 as described above in reference to FIG. 2. In an embodiment of the present approach, a level of confidence in the accuracy of the horizontal edge information 104 may be determined based on the resolution of the ultrasound dataset and other factors. This horizontal edge information 104 may then be combined with at least one tomosynthesis dataset 98 to reconstruct a dataset with improved horizontal edge information in a step 106. In one embodiment, the confidence level of the horizontal edge information 104 may contribute to how much weight the information 104 is given in the reconstruction step 106. A jointly reconstructed dataset 108 may then be post-processed in a step 110. A jointly reconstructed three-dimensional image 114 may then be produced.

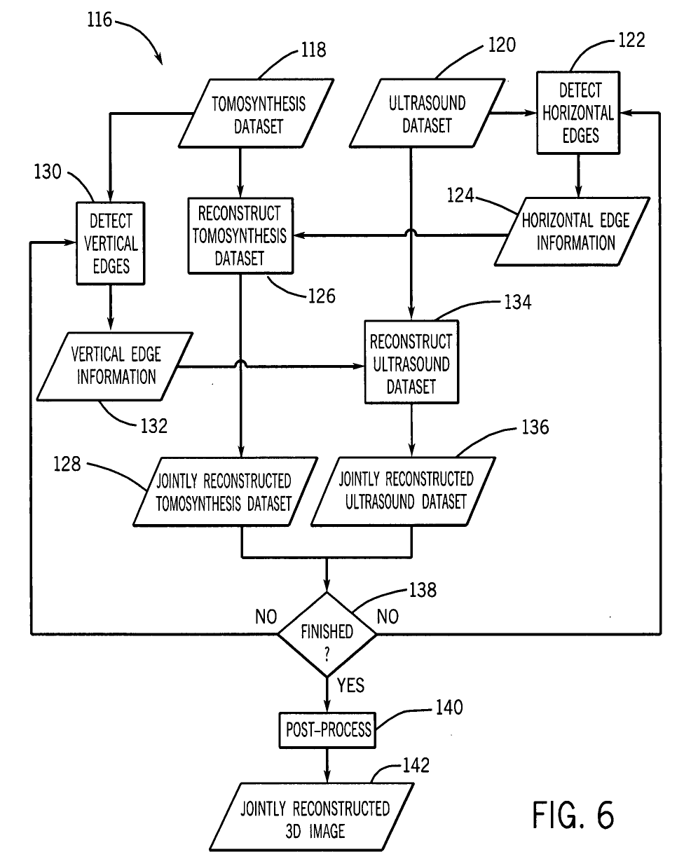

In accordance with another embodiment of the present approach, a process 116 is illustrated in FIG. 6. As in the embodiment described in reference to FIG. 5, at least one ultrasound dataset 120 may be analyzed to detect horizontal edges in a step 122. Confidence levels for the detected horizontal edges may also be determined. Horizontal edge information 124 with higher confidence levels may be given more weight and edges with lower confidence levels may be given less weight or disregarded in reconstruction step 126. A jointly reconstructed tomosynthesis dataset 128 may be reconstructed from at least one tomosynthesis dataset 118 using, for example, Markov random fields (MRF) or similar techniques in a step 126, where the horizontal edge information 124 may be injected as a local smoothness constraint or lack thereof. This constraint may also reflect the confidence associated with the different edge locations. The algorithm for the reconstruction step 126 may encourage “smooth” behavior, except in locations where the dataset 118 or the horizontal edge information 124 does not support this assumption.

In a parallel track of process 116, the tomosynthesis dataset 118 may be analyzed to detect vertical edges in a step 130. In this embodiment, “vertical” is in a direction generally along the X-ray beam, as described in reference to FIG. 1. In practice, the “vertical” plane will generally be perpendicular to the X-ray detector 16 and compression plates 20 as described above in reference to FIG. 1. In addition, confidence levels for the detected vertical edges may be determined. The derived vertical edge information 132 and the associated confidence levels may then be combined with the registered ultrasound dataset 120 to produce a jointly reconstructed ultrasound dataset 136 in a reconstruction step 134. Steps 122 through 134 may then be repeated until further iterations fail to yield substantial improvements in the jointly reconstructed datasets 128 and 136. Finally, the jointly reconstructed datasets 128 and 136 may undergo post-processing in a step 140. A jointly reconstructed three-dimensional image 142 may then be displayed. The datasets 128 and 136 may be a single combined multi-parameter (or multi-modality) dataset, which reflects both tomosynthesis and ultrasound characteristics. Reconstruction steps 126 and 134 may also be a combined step that utilizes information from the tomosynthesis dataset 118 and ultrasound dataset 120, as well as the previously-estimated multi-parameter datasets 128 and/or 136, in an iterative process.

In another embodiment, edge information may be extracted jointly from both datasets 118 and 120, where the confidence levels for one edge orientation are higher in one modality and the confidence levels for another edge orientation are higher in another modality. For example, objective function based approaches may be used, where the objective function reflects the different confidence levels. The objective function may be minimized or maximized, depending on the formulation. An exemplary objective function approach may use active contours, or snakes, as known in the literature. This approach may also incorporate prior information about the imaged anatomy, such as from an atlas. For example, the atlas may be registered to the imaged anatomy, and the initial estimate of the location of edges may be derived from the atlas.

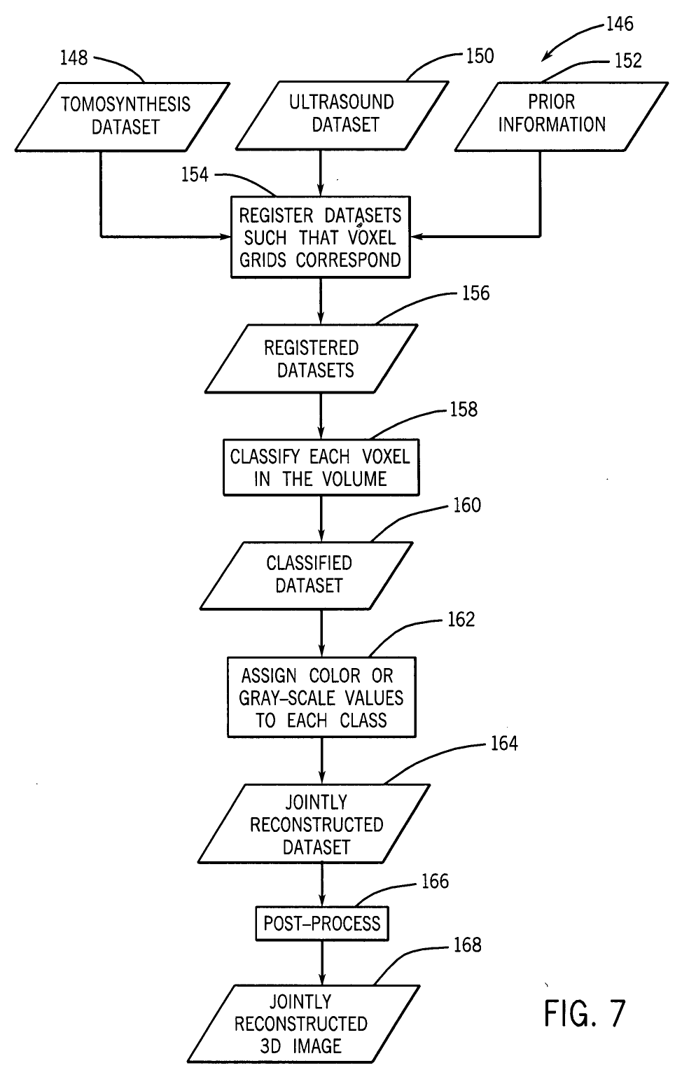

Turning now to FIG. 7, in an exemplary embodiment of the present approach, at least one tomosynthesis dataset 148 and at least one ultrasound dataset 150 may be combined with prior information 152 in a step 154 to produce registered datasets 156. In this context, prior information 152 may include an anatomical atlas, such as, for example, geometrical shape models, models of tissue distribution, or models of tissue composition within certain regions of the anatomy or of the image. Alternatively, the prior information 152 may include other images of the anatomy, such as, for example, a CT scan, a MR scan, or previous tomosynthesis or ultrasound scans. According to an embodiment of the present approach, the prior information 152 may include only a subset of a structures or descriptive information. For example, prior information 152 may include a constraint that the X-ray attenuation values only correspond to two values, those of fatty tissue and fibroglandular tissue. In one embodiment of the present approach, the tomosynthesis and ultrasound datasets 148 and 150 may be intrinsically registered and registration step 154 may be merely used to align the datasets with the prior information 152.

In a step 158, information from the datasets 156 registered to the prior information 152 may be used to classify each voxel in the imaged volume, creating a classified dataset 160. That is, each voxel may be assigned a value or label based on information obtained from two or more of the tomosynthesis imaging, the ultrasound imaging, and the prior knowledge gained from the anatomical atlas. For example, based on models of tissue distribution, the subcutaneous fat layer may be easily identifiable in the ultrasound dataset, and this information may flow directly into the joint reconstruction. Alternatively, techniques from multi-sensor fusion may be applied to classify the volume in step 158 in accordance with an embodiment of the present approach. That is, each voxel in the registered datasets 156 may be placed into a class, such as, for example, fat, fibroglandular tissue, or calcifications. The classes could also contain anatomical information, such as subcutaneous fat layer, duct, Cooper's ligaments, etc. The classification may, for example, be based on two or more of the ultrasound dataset, the raw ultrasound data, the X-ray projections, the tomosynthesis reconstruction, the prior information, and a first stage classification which may have acted on a reduced set of data and which may have an associated confidence level. Once the combined datasets have been classified, color or gray-scale values may be assigned to each class in a step 162. The jointly reconstructed dataset 164 may then be post-processed in a step 166 to produce a jointly reconstructed three-dimensional image 168.

Prior information 152 may also be used in other embodiments of the present technique, such as, for example, reconstruction steps 126 and 134 of process 116, illustrated in FIG. 6. In process 116, prior information 152 may be utilized to assign class information to voxels in the reconstructed datasets 128 and 136.

In addition, the objective function based approach described in relation to FIG. 6 may be applied to the process 146 of FIG. 7. For example, the objective function may contain penalty terms for class membership, smoothness, length of edges between regions, or other classification information. While FIG. 6 refers to an embodiment employing primarily edge-based segmentation and reconstruction, FIG. 7 refers to an embodiment employing primarily region-based segmentation and reconstruction. Combined, hybrid approaches may also be used. Furthermore, the registration step may also be performed in conjunction with the multi-modality reconstruction, in an integrated processing step.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A method for reconstructing an imaging dataset comprising:
providing at least a first dataset acquired via a first imaging technique or a first image generated from the first dataset;
providing at least a second dataset acquired via a second imaging technique or a second image generated from the second dataset, wherein the first imaging technique has better resolution than the second imaging technique in at least a first direction and the second imaging technique has better resolution than the first imaging technique in at least a second direction; and
generating a volumetric dataset by extracting information from the first dataset or the first image and the second dataset or the second image.

2. The method of claim 1, comprising generating an image from the volumetric dataset.

3. The method of claim 1, wherein the first and second imaging techniques comprise different imaging modalities.

4. The method of claim 1, wherein the first and second imaging techniques comprise different imaging orientations.

5. The method of claim 1, wherein generating a volumetric dataset comprises:
deriving a mapping function based on similarities between the first dataset or the first image and the second dataset or the second image;
assigning the one or more intensity or color values attributable to the first technique to the one or more intensity or color values attributable to the second technique based on the mapping function; and
processing the datasets or the images to generate an image according to the mapped intensity or color values.

6. The method of claim 5, wherein deriving a mapping function comprises a point-by-point, a pixel-by-pixel, a voxel-by-voxel, a region-by-region, or a subset-by-subset comparison between the first dataset or the first image and the second dataset or the second image.

7. The method of claim 5, wherein deriving a mapping function comprises:
dividing the first dataset or the first image into a plurality of subsets;
projecting the plurality of subsets to form a plurality of basis images;
approximating the second dataset or the second image as a linear combination of the plurality of basis images; and
deriving one or more representative intensity or color values attributable to the second technique from the linear combination of the plurality of basis images.

8. The method of claim 7, wherein dividing the first dataset or the first image into the plurality of subsets comprises grouping voxels of the first dataset or the first image according to the intensity or color values attributable to the first imaging technique or segmenting the first dataset or the first image into homogeneous regions.

9. The method of claim 8, wherein segmenting comprises using an oversegmentation method such that there is a high confidence that the data within each region is homogeneous.

10. The method of claim 1, wherein generating a volumetric dataset comprises segmenting at least the first dataset or the first image using at least one of an edge-based segmentation method, a region-based segmentation technique, or an objective-function based segmentation technique.

11. The method of claim 1, wherein generating a volumetric dataset comprises deriving a mapping function by classifying voxels in a volume of interest based on information obtained from two or more of the first dataset or the first image, the second dataset or the second image, and an anatomical atlas.

12. The method of claim 1, wherein generating a volumetric dataset comprises detecting information about one or more edges in at least one dataset or image.

13. The method of claim 1, wherein generating a volumetric dataset comprises:
detecting information about one or more edges in the first direction from the first dataset or the first image;
inputting the information about the one or more edges in the first direction into a reconstruction algorithm; and
generating the volumetric dataset from at least the second dataset or the second image using the reconstruction algorithm.

14. The method of claim 13, wherein the information about the one or more edges is input into the reconstruction algorithm as a local smoothness constraint or a lack of local smoothness.

15. The method of claim 13, comprising:
determining confidence levels for the information about the one or more edges; and
inputting the confidence levels into the reconstruction algorithm.

16. The method of claim 1, comprising registering the first dataset or the first image and the second dataset or the second image.

17. The method of claim 1, comprising processing the volumetric dataset to recover fine-scale information.

18. One or more tangible, machine readable media, comprising code executable to perform the act of generating a volumetric dataset by extracting information from a first dataset acquired using a first imaging technique or a first image generated from the first dataset and a second dataset acquired using a second imaging technique or a second image generated from the second dataset, wherein the first imaging technique has better resolution than the second imaging technique in at least a first direction and the second imaging technique has better resolution than the first imaging technique in at least a second direction.

19. The tangible, machine readable media of claim 18, comprising code executable to perform the act of generating an image from the volumetric dataset.

20. The tangible, machine readable media of claim 18, comprising code executable to perform the act of generating a volumetric dataset by:
deriving a mapping function based on similarities between the first dataset or the first image and the second dataset or the second image;
assigning the one or more intensity or color values attributable to the first technique to the one or more intensity or color values attributable to the second technique based on the mapping function; and
processing the datasets or the images to generate an image according to the mapped intensity or color values.

21. The tangible, machine readable media of claim 20, comprising code executable to perform the act of deriving a mapping function based on a point-by-point, a pixel-by-pixel, or a voxel-by-voxel comparison between the first dataset or the first image and the second dataset or the second image.

22. The tangible, machine readable media of claim 20, comprising code executable to perform the act of deriving a mapping function by:
dividing the first dataset or the first image into a plurality of subsets;
projecting the plurality of subsets as a plurality of basis images;
approximating the second dataset or the second image as a linear combination of the plurality of basis images; and
deriving one or more representative intensity or color values attributable to the second technique from the linear combination of the plurality of basis images.

23. The tangible, machine readable media of claim 18, comprising code executable to perform the act of generating a volumetric dataset by classifying voxels in a jointly reconstructed dataset based on information obtained from two or more of the first dataset or the first image, the second dataset or the second image, and an anatomical atlas.

24. The tangible, machine readable media of claim 18, comprising code executable to perform the act of generating a volumetric dataset by:
detecting information about one or more edges in the first direction from the first dataset or the first image;
inputting the information about the one or more edges in the first direction into a reconstruction algorithm; and
generating a volumetric dataset from at least the second dataset or the second image using the reconstruction algorithm.

25. The tangible, machine readable media of claim 24, comprising code executable to perform the act of generating a volumetric dataset by:
detecting information about one or more edges in the second direction from the second dataset or the second image;
inputting the information about the one or more edges in the second direction into the reconstruction algorithm; and
generating the volumetric dataset from the first dataset or the first image and the second dataset or the second image using the reconstruction algorithm.

26. An image processing system comprising:
a computer, wherein the computer is configured to generate a volumetric dataset by extracting information from a first dataset acquired using a first imaging technique or a first image generated from the first dataset and a second dataset acquired using a second imaging technique or a second image generated from the second dataset, wherein the first imaging technique has better resolution than the second imaging technique in at least a first direction and the second imaging technique has better resolution than the first imaging technique in at least a second direction.

27. The image processing system of claim 26, wherein the computer is further configured to generate an image from the volumetric dataset.

28. The image processing system of claim 26, comprising memory configured to store the first dataset, the first image, the second dataset, the second image, the volumetric dataset, routines executable by the computer for performing the generation, or a combination thereof.

29. The image processing system of claim 26, comprising an operator workstation and display for viewing the first dataset, the first image, the second dataset, the second image, the generated volumetric dataset, or a combination thereof.
